(19) United States
(12) Patent Application Publication
(10) Pub. No.: US 2024/0398476 A1
(43) Pub. Date: Dec. 5, 2024

(54) MEASUREMENT AND DIAGNOSTIC SYSTEM FOR WEIGHT-BEARING LINES OF LOWER LIMBS AND INTELLIGENT MEASUREMENT METHOD THEREOF

(71) Applicant: TIANJIN HOSPITAL, Tianjin (CN)

(72) Inventors: Xinlong MA, Tianjin (CN); Jianxiong MA, Tianjin (CN); Zhichao WU, Tianjin (CN); Xinjun ZHU, Tianjin (CN); Haohao BAI, Tianjin (CN); Ying WANG, Tianjin (CN); Bin LU, Tianjin (CN); Lei SUN, Tianjin (CN); Hongzhen JIN, Tianjin (CN)

(21) Appl. No.: 18/203,432

(22) Filed: May 30, 2023

(51) Int. Cl. A61B 34/10

(52) U.S. Cl. CPC A61B 34/10; A61B 2034/105; A61B 2034/108

(57) ABSTRACT

The present disclosure discloses a measurement and diagnostic system for weight-bearing lines of lower limbs. The measurement and diagnostic system includes a data loading module, a keypoint selection module, an angle calculation module and a mechanical axis diagnosis and output module, wherein the angle calculation module is configured to calculate slopes among keypoints by using obtained keypoints, characteristic axes and joint lines, and calculate mechanical lateral distal femoral angles, joint line convergence angles, medial proximal tibial angles and lateral distal tibial angles by inverse trigonometric functions; and the mechanical axis diagnosis and output module is configured to compare the obtained angle values with preset values to judge whether a lower limb deformity occurs. The present disclosure further discloses an intelligent measurement method for weight-bearing lines of both lower limbs. The precise coordinates of the keypoints are not required in the present disclosure.

### TECHNICAL FIELD

The present disclosure relates to the field of human body keypoint identification based on neural networks, in particular to a diagnostic system and an intelligent measurement method for weight-bearing lines of both lower limbs.

### BACKGROUND ART

A lower limb deformity is a common orthopedic disease in clinic, including various pathogenies, e.g., osteoarthritis, rhachitis, and traumatic fracture malunion. As reported in the literature, the prevalence of osteoarthritis is up to 10% in men and 18% in women amongst elderly people aged 60 or above. The lower limb deformity not only has impact on appearance of patients, but also is accompanied with articular pain in severe cases to affect joint functions, causing physical and psychological harm to patients and reducing the quality of life. Thus, it is very important to perform a corrective surgery for the severe lower limb deformity in time.

In the corrective surgery for the lower limb deformity, accurate diagnosis of deformity and planning of mechanical axes are the key factors to determine the success of the surgery, which need to draw mechanical axes of lower limbs and measure key angles on full-length radiographs of lower limbs by a doctor. At present, the most widely used measurement in clinic is manual measurement, which needs to manually mark points, draw lines and measure angles on full-length radiographs of lower limbs by a surgeon. However, manual measurement has larger subjective errors and low diagnostic efficiency due to influences of human factors such as doctor level and mental state.

Some measurement software such as OsteoMaster is available on the market. Radiographs are imported into the software to plan corresponding points and lines manually, to obtain corresponding angles automatically. Manual intervention is reduced to a certain extent, but large subjective errors still exist. The accurate measurement of the mechanical axes of the lower limbs is directly related to improvement of surgical efficacy. Nowadays, there are a large number of cases in orthopedics clinically. Orthopedists are under much pressure. Though it is not difficult to draw and measure the mechanical axes and corresponding angles of the lower limbs, it is more complicated, and wastes medical resources greatly.

Therefore, a diagnostic system and an intelligent measurement method for weight-bearing lines of both lower limbs are of great significance, which features simple operation and accurate measurement of mechanical axes of both lower limbs.

### SUMMARY

The present disclosure aims to provide a measurement and diagnostic system for weight-bearing lines of lower limbs and an intelligent measurement method thereof. The present disclosure does not need precise coordinates of keypoints. For knee joint orientation lines and ankle joint lines, by replacing point coordinates with slopes, full-length images of human lower limbs are processed by deep learning technologies such as convolutional neural networks, and digital image processing technologies, to quickly and accurately achieve drawing and calculation of mechanical axes and joint angles of lower limbs.

A measurement and diagnostic system for weight-bearing lines of both lower limbs includes:
a data loading module, configured to load and read a data file (.mat) of keypoint position information of training sets and radiographs to be measured, and select joint image positions in each radiograph;
a keypoint selection module, configured to classify and identify the joint image positions selected by the data loading module through a convolutional neural network by taking obtained joint images as input layers, to obtain coordinates of corresponding keypoints on the radiographs, including coordinates of eight characteristic axis keypoints and coordinates of a plurality of joint line keypoints; connect the obtained coordinates of the eight keypoints to draw characteristic axes, thereby forming four characteristic axes extending along left and right lower limbs, including a first characteristic axis, a second characteristic axis, a third characteristic axis and a fourth characteristic axis; and connect the coordinates of the joint line keypoints to draw six joint lines, thereby forming left and right knee joint upper orientation lines, left and right knee joint lower orientation lines, and left and right ankle joint orientation lines, wherein the first and second characteristic axes correspond to the left and right knee joint upper orientation lines, and the third and fourth characteristic axes correspond to the left and right knee joint lower orientation lines, and the left and right ankle joint orientation lines;
an angle calculation module, configured to calculate slopes among the keypoints by using the obtained keypoints, characteristic axes and joint lines, and calculate mechanical lateral distal femoral angles (mLDFA), joint line convergence angles (JLCA), medial proximal tibial angles (MPTA) and lateral distal tibial angles (LDTA) by inverse trigonometric functions; and
a mechanical axis diagnosis and output module, configured to compare obtained angle values with preset reference thresholds of the mechanical lateral distal femoral angles, the joint line convergence angles, the medial proximal tibial angles and the lateral distal tibial angles according to the angles obtained by the angle calculation module, wherein if the obtained angle values are not within the reference thresholds, it indicates that the angles are abnormal, and there is possibly a lower limb deformity, and if all the angle values are within the reference thresholds, it indicates that there is no lower limb deformity; and then, output the obtained angle values, show the plurality of characteristic axes and joint lines on the radiographs, and represent measurement results in a picture output form.

Furthermore, the preset reference thresholds of the mechanical lateral distal femoral angles, the joint line convergence angles, the medial proximal tibial angles and the lateral distal tibial angles are as follows: the reference threshold is 85° to 90° for the mechanical lateral distal femoral angles, 0° to 2° for the joint line convergence angles, 85° to 90° for the medial proximal tibial angles, and 86° to 92° for the lateral distal tibial angles.

Furthermore, the joint images include a left hip joint area, a right hip joint area, a left knee joint area, a right knee joint area, a left ankle joint area and a right ankle joint area;
the characteristic axis keypoints include a first keypoint, a second keypoint, a third keypoint, a fourth keypoint, a fifth keypoint, a sixth keypoint, a seventh keypoint and an eighth keypoint;
the first keypoint is a center point of a left femoral head in the left hip joint area, the second keypoint is a center point of a right femoral head in the right hip joint area, the third keypoint is a center point of a left femoral knee joint in the left knee joint area, the fourth keypoint is a center point of a left tibial knee joint in the left knee joint area, the fifth keypoint is a center point of a right femoral knee joint in the right knee joint area, the sixth keypoint is a center point of a right tibial knee joint in the right knee joint area, the seventh keypoint is a center point of a left ankle joint in the left ankle joint area, and the eighth keypoint is a center point of a right ankle joint in the right ankle joint area;
wherein two endpoints of the first characteristic axis are the first keypoint and the third keypoint respectively, two endpoints of the second characteristic axis are the second keypoint and the fifth keypoint respectively, two endpoints of the third characteristic axis are the fourth keypoint and the seventh keypoint respectively, and two endpoints of the fourth characteristic axis are the sixth keypoint and the eighth keypoint respectively.

Furthermore, the left and right knee joint upper orientation lines, the left and right knee joint lower orientation lines, and the left and right ankle joint orientation lines are obtained as follows:
the joint line keypoints include lowest points of medial and lateral femoral condyles at a distal end of a left femur in the left knee joint area, lowest points of medial and lateral femoral condyles at a distal end of a right femur in the right knee joint area, lowest points of medial and lateral tibial plateaus at a proximal end of a left tibia in the left knee joint area, lowest points of medial and lateral tibial plateaus at a proximal end of a right tibia in the right knee joint area, lowest points of medial and lateral articular surfaces at a distal end of the left tibia in the left ankle joint area, and lowest points of medial and lateral articular surfaces at a distal end of the right tibia in the right ankle joint area;
the left knee joint upper orientation line is a connecting line between two joint line keypoints located at the distal end of the left femur;
the right knee joint upper orientation line is a connecting line between two joint line keypoints located at the distal end of the right femur;
the left knee joint lower orientation line is a connecting line between two joint line keypoints located at the proximal end of the left tibia;
the right knee joint lower orientation line is a connecting line between two joint line keypoints located at the proximal end of the right tibia;
the left ankle joint orientation line is a connecting line between two joint line keypoints located at the distal end of the left tibia; and
the right ankle joint orientation line is a connecting line between two joint line keypoints located at the distal end of the right tibia.

An intelligent measurement method for weight-bearing lines of both lower limbs includes the following steps:
step 1, importing a plurality of human lower limb full-length digital radiographs to be measured, loading and reading keypoint position training models pre-trained properly, and selecting joint image positions in each radiograph, wherein joint images include a left hip joint area, a right hip joint area, a left knee joint area, a right knee joint area, a left ankle joint area and a right ankle joint area;
step 2, extracting keypoints by a convolutional neural network method
performing classification and identification through a convolutional neural network by taking the joint images obtained in step 1 as input layers, to obtain coordinates of the corresponding keypoints on the radiographs, including coordinates of eight characteristic axis keypoints and coordinates of a plurality of joint line keypoints; connecting the obtained coordinates of the eight keypoints to draw characteristic axes, thereby forming four characteristic axes extending along left and right lower limbs, including a first characteristic axis, a second characteristic axis, a third characteristic axis and a fourth characteristic axis; and connecting the coordinates of the joint line keypoints to draw six joint lines, thereby forming left and right knee joint upper orientation lines, left and right knee joint lower orientation lines, and left and right ankle joint orientation lines, wherein the first and second characteristic axes correspond to the left and right knee joint upper orientation lines respectively, and the third and fourth characteristic axes correspond to the left and right knee joint lower orientation lines, and the left and right ankle joint orientation lines respectively; and
the characteristic axis keypoints include a first keypoint, a second keypoint, a third keypoint, a fourth keypoint, a fifth keypoint, a sixth keypoint, a seventh keypoint and an eighth keypoint;
step 3, calculating slopes among the keypoints by using the obtained keypoints, characteristic axes and joint lines, and calculating mechanical lateral distal femoral angles, joint line convergence angles, medial proximal tibial angles and lateral distal tibial angles by inverse trigonometric functions; wherein
the mechanical lateral distal femoral angles (mLDFA) include an included angle between the first characteristic axis and the left knee joint upper orientation line where the third keypoint is located, and an included angle between the second characteristic axis and the right knee joint upper orientation line where the fifth keypoint is located; a calculation formula of the included angles is as follows:

$$tan θ = \vert \frac{{k}_{13} - {k}_{CD}}{1 + {k}_{13} {k}_{CD}} \vert = \vert \frac{\frac{{y}_{3} - {y}_{1}}{{x}_{3} - {x}_{1}} - \frac{{y}_{D} - {y}_{C}}{{x}_{D} - {x}_{C}}}{1 + \frac{{y}_{3} - {y}_{1}}{{x}_{3} - {x}_{1}} \cdot \frac{{y}_{D} - {y}_{C}}{{x}_{D} - {x}_{C}}} \vert$$

Where, θ represents the solved angle, k₁₃ represents a slope of the first characteristic axis, k_{CD} represents a slope of the left knee joint upper orientation line, and x₁ and y₁ represent an abscissa and an ordinate of the first keypoint; x₃ and y₃ represent an abscissa and an ordinate of the third keypoint; x_{C}, x_{D}, y_{C} and y_{D} represent abscissas and ordinates of two joint line keypoints on the left knee joint upper orientation line; the function is transformed into an arctan that is a reciprocal function according to the above tangent formula, to solve the left mechanical lateral distal femoral angle; correspondingly, the included angle between the second characteristic axis and the right knee joint upper orientation line where the fifth keypoint is located may be obtained according to the above formula;
the joint line convergence angles (JLCA) include an included angle between the left knee joint upper orientation line where the third keypoint is located, and the left knee joint lower orientation line where the fourth keypoint is located, and an included angle between the right knee joint upper orientation line where the fifth keypoint is located, and the right knee joint lower orientation line where the sixth keypoint is located; calculation steps of the included angles apply the calculation formula of the mechanical lateral distal femoral angles;
the medial proximal tibial angles (MPTA) include an included angle between the left knee joint lower orientation line where the fourth keypoint is located, and the third characteristic axis, and an included angle between the right knee joint lower orientation line where the sixth keypoint is located, and the fourth characteristic axis; calculation steps of the included angles apply the calculation formula of the mechanical lateral distal femoral angles;
the lateral distal tibial angles (LDTA) include an included angle between the third characteristic axis and the left ankle joint orientation line where the seventh keypoint is located, and an included angle between the fourth characteristic axis and the right ankle joint orientation line where the eighth keypoint is located; calculation steps of the included angles apply the calculation formula of the mechanical lateral distal femoral angles; and
step 4, outputting and storing results
comparing the angle values obtained in step 3 with preset reference thresholds of the mechanical lateral distal femoral angles, the joint line convergence angles, the medial proximal tibial angles and the lateral distal tibial angles, wherein if the obtained angle values are not within the reference thresholds, it indicates that the angles are abnormal, and there is possibly a lower limb deformity, and if all the angle values are within the reference thresholds, it indicates that there is no lower limb deformity; and then, outputting the obtained angle values, showing the plurality of characteristic axes and joint lines on the radiographs, and representing measurement results in a picture output form.

Furthermore, the preset reference thresholds of the mechanical lateral distal femoral angles, the joint line convergence angles, the medial proximal tibial angles and the lateral distal tibial angles are as follows: the reference threshold is 85° to 90° for the mechanical lateral distal femoral angles, 0° to 2° for the joint line convergence angles, 85° to 90° for the medial proximal tibial angles, and 86° to 92° for the lateral distal tibial angles.

Furthermore, the keypoint position training module is configured to train a keypoint model by a target detection algorithm model in advance, to obtain specific positions of all keypoints in the radiographs; and the keypoint position information is saved by the training model properly trained in a .mat format for extracting keypoints subsequently.

Furthermore, the step 4 further includes analyzing errors of the angle values obtained in step 3, specifically including:
regarding it as a multivariate function θ=arctan (xₙ) according to a trigonometric formula for calculating the included angles, letting the function in brackets be f, solving a partial derivative, substituting the function f, and simplifying it to obtain the following error analysis formula of abscissas and ordinates of characteristic axis keypoints on a single radiograph:

$$\frac{\partial θ}{\partial {x}_{n}} = \frac{1}{1 + {f}^{2}} \frac{\partial f}{\partial {x}_{n}} \\ \frac{\partial θ}{\partial {y}_{n}} = \frac{1}{1 + {f}^{2}} \frac{\partial f}{\partial {y}_{n}}$$

Where, xₙ is an abscissa of a characteristic axis keypoint, and yₙ is an ordinate of the characteristic axis keypoint.

Error values of the characteristic axis keypoints are obtained according to the above formula in sequence; and statistical analysis results of the error values obtained from a plurality of radiographs are drawn into a line chart for analysis by users.

Furthermore, the step 4 further includes recording the angle values obtained in step 3in pre-initialized Excel.

Compared with the prior art, the technical solution of the present disclosure has the following beneficial effects:
the full-length images of human lower limbs are processed by deep learning technologies such as convolutional neural networks, and digital image processing technologies based on an anatomical theory in conjunction with a morphological characteristic identification and extraction technology in an imaging field, to complete fitting of a femoral head center, extraction of a knee joint boundary, fitting of a boundary line, extraction of intersection points and the like in weight-bearing radiographs of both lower limbs, and quickly and accurately achieve drawing and calculation of mechanical axes and joint angles of the lower limbs;
considering that the coordinate errors of the keypoints are difficult to decrease, the precise coordinates of the keypoints are not required in the present disclosure; for the knee joint orientation lines and the ankle joint lines, by replacing point coordinates with the slopes, the angles are calculated by an arctan inverse trigonometric function to reduce the angle calculation error obviously;
according to the present disclosure, the complicated deformity screening work can be done, and diagnosis-related information is quickly extracted from image data to shorten the diagnostic operation duration and improve the utilization rate of medical resources; the success rate of the corrective surgery for the lower limb deformity can also be improved indirectly by the system, to avoid undercorrection or overcorrection of the deformity caused by diagnostic errors, and reduce a re-operation risk and decrease the economic burden of a patient; and the precise and intelligent correction of an orthopaedic deformity in clinic is achieved, medical risks are effectively reduced, and the quality of medical care is improved basically.

### BRIEF DESCRIPTION OF THE DRAWINGS

FIG. 1 is a flow chart of an intelligent measurement system for weight bearing lines of both lower limbs according to the present disclosure;

FIG. 2 shows radiographs of lower limbs in different patterns with measurement results marked under collection of an experimental data set in an embodiment of the present disclosure; wherein, FIG. 2A is a radiograph of lower limbs of a patient A, FIG. 2B is a radiograph of lower limbs of a patient B, FIG. 2C is a radiograph of lower limbs of a patient C, and FIG. 2D is a radiograph of lower limbs of a patient D; FIG. 3 is a line chart of statistical analysis results of errors obtained in step 4;

FIG. 4 is a flow chart of an intelligent measurement method for weight bearing lines of lower limbs according to the present disclosure;

FIG. 5 is a structural diagram of a convolutional neural network used in step 2; and

FIG. 6A shows left knee joint upper and lower orientation lines; and FIG. 6B shows a left ankle joint orientation line.

In the drawings

| | |
|---|---|
| 1. First keypoint | 2. Second keypoint |
| 3. Third keypoint | 4. Fourth keypoint |
| 5. Fifth keypoint | 6. Sixth keypoint |
| 7. Seventh keypoint | 8. Eighth keypoint |
| 9. First characteristic axis | 10. Second characteristic axis |
| 11. Third characteristic axis | 12. Fourth characteristic axis |
| 13. Knee joint upper orientation | 14. Knee joint lower orientation |
| line | line |
| 15. Ankle joint orientation line | |

### DETAILED DESCRIPTION OF THE PRESENT DISCLOSURE

The technical solution of the present disclosure will be described below in detail with reference to accompanying drawings and specific embodiments. The described specific embodiments are not intended to limit the present disclosure, but explain it.

Embodiments of the present disclosure provide a measurement and diagnostic system and an intelligent measurement method for weight-bearing lines of both lower limbs. According to the present disclosure, the complicated deformity screening work can be done based on the anatomical theory in conjunction with the morphological characteristic identification and extraction technology in an imaging field, and diagnosis-related information is quickly extracted from image data of radiographs to shorten the diagnostic operation duration and improve the utilization rate of medical resources; and the success rate of the corrective surgery for the lower limb deformity can also be improved indirectly by the system, to avoid undercorrection or overcorrection of the deformity caused by diagnostic errors and reduce a re-operation risk.

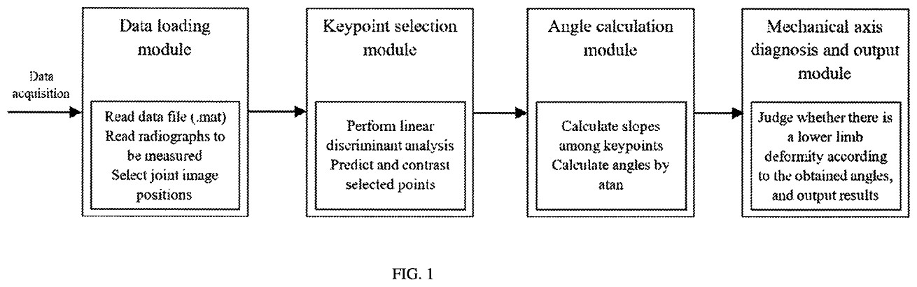

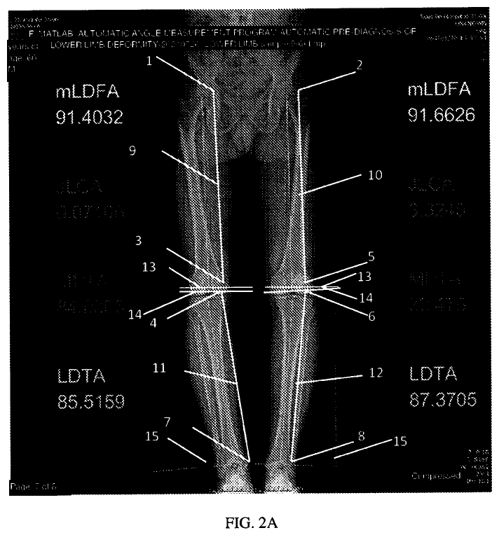

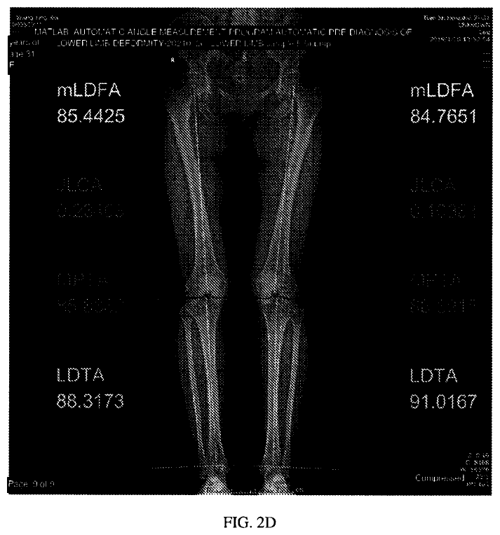

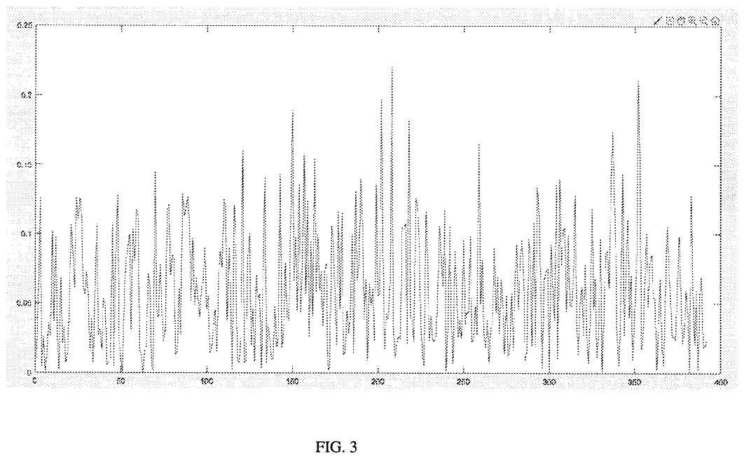

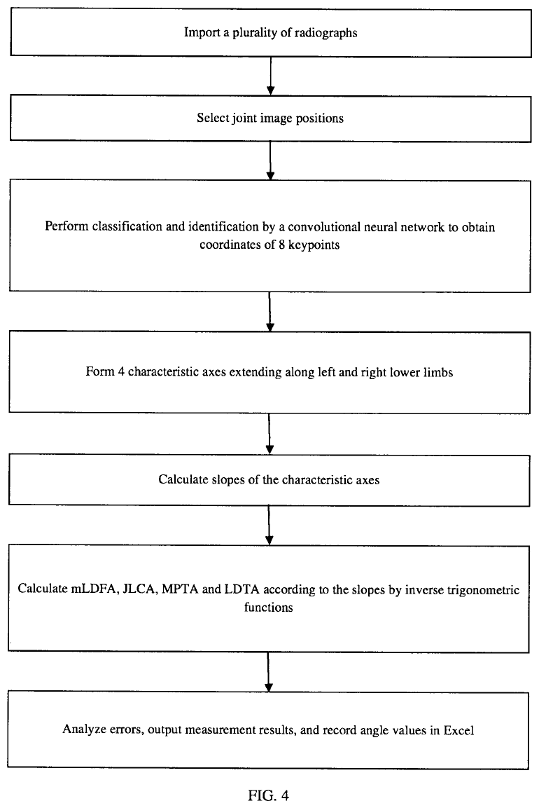

As shown in FIG. 1, a measurement and diagnostic system for weight-bearing lines of both lower limbs includes a data loading module, a keypoint selection module, an angle calculation module and a mechanical axis diagnosis and output module. The specific operations of the modules include the following steps as shown in FIG. 4:

### Step 1: Data Training and Loading

a plurality of complete full-length weight-bearing radiographs of lower limbs collected by a picture archiving and communication system (PACS) in Tianjin Hospital, on which keypoints may be clearly identified, are selected in advance, keypoint position information (Ground Truth) in each radiograph is marked by a ginput () function of MATLAB, and images of various lower limbs in different patterns and correspondingly marked keypoint position information serve as experimental data sets in the present disclosure. The experimental data sets are imported into a target detection algorithm model for model training, and specific positions of all keypoints on the radiographs are obtained after repeated training; and the training model properly trained is saved in a .mat format for subsequent image processing training and extraction of keypoint positions in practical application.

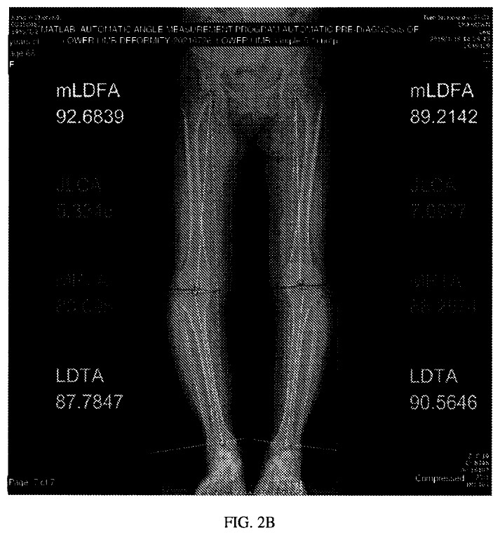

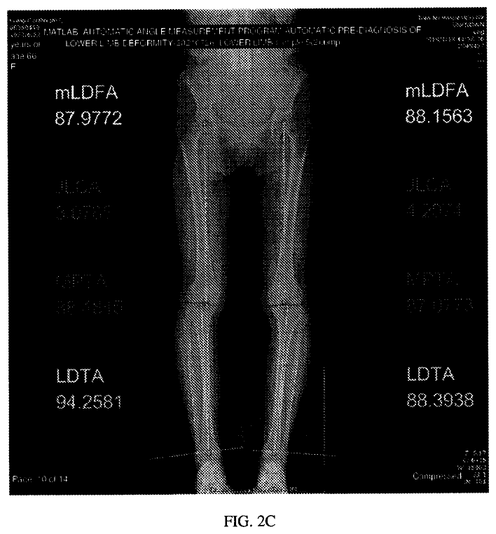

The .mat files of the keypoint position information of training sets are loaded and read, and Excel is initialized for recording results; a plurality of to-be-measured full-length digital radiographs (hereafter referred to as radiographs) of human lower limbs of patients A, B, C and D shown in FIGS. 2A, 2B, 2C and 2D are read cyclically in sequence, and each radiograph is numbered and designated. Then, as shooting standards of the radiographs are consistent, the position of each joint on the radiographs is not different greatly; two-dimensional plane coordinate systems of the read radiographs are established automatically, and joint image positions in each radiograph are selected by a fixed area positioning method in a manner of determining a fixed selective area with four fixed coordinate points, wherein the joint images include a left hip joint area, a right hip joint area, a left knee joint area, a right knee joint area, a left ankle joint area and a right ankle joint area.

### Step 2: Keypoint Selection

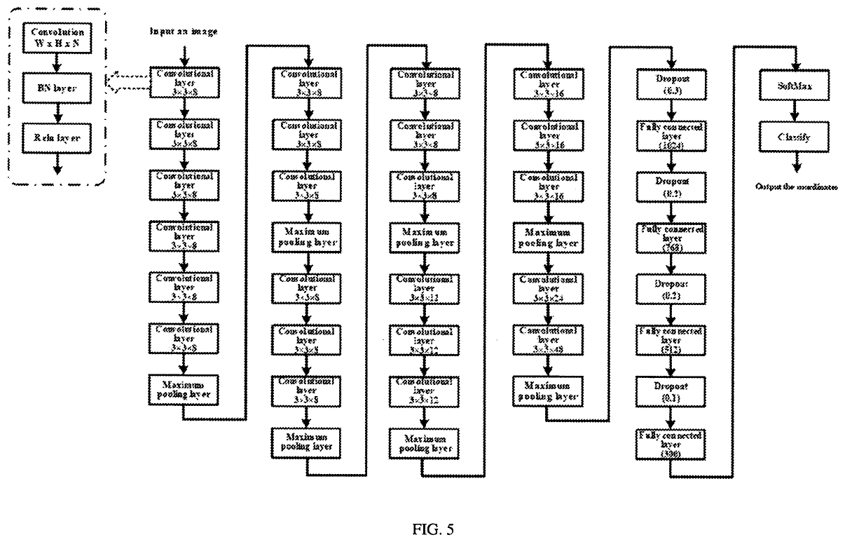

S201: as the joint image areas obtained in step 1 are intercepted from the radiographs with consistent pixels, characteristic matrices of the areas are the same. Different joint images of both lower limbs are respectively classified and identified by a convolutional neural network; linear discriminant analysis is conducted firstly, and then prediction and contrast of selected points are performed to obtain keypoints. Specifically, as shown in FIG. 5, six joint image areas obtained in step 1 serve as input layers respectively for operation by a convolutional neural network method; firstly, convolution operation is performed by a convolution kernel of 3*3*8 to obtain a maximum pooling layer, and then dropout is performed after 5 times of convolution; all final data is linearly transformed on a fully connected layer, and prediction tags of the areas are output on a softmax layer thereafter; the linearly transformed results are mapped to a corresponding category to achieve classification of coordinates, and coordinates of the corresponding keypoints on the radiographs are output; and the data is temporarily stored by variables in a double format for subsequent calculation of the angles. The coordinates of a total of 8 characteristic axis keypoints and the coordinates of 12 joint line keypoints are provided in the embodiment.

Wherein, the 8 characteristic axis keypoints include a first keypoint 1, a second keypoint 2, a third keypoint 3, a fourth keypoint 4, a fifth keypoint 5, a sixth keypoint 6, a seventh keypoint 7 and an eighth keypoint 8. The first keypoint 1 is a center point of a left femoral head of the left hip joint area, the second keypoint 2 is a center point of a right femoral head in the right hip joint area, the third keypoint 3 is a center point of a left femoral knee joint in the left knee joint area, the fourth keypoint 4 is a center point of a left tibial knee joint in the left knee joint area, the fifth keypoint 5 is a center point of a right femoral knee joint in the right knee joint area, the sixth keypoint 6 is a center point of a right tibial knee joint in the right knee joint area, the seventh keypoint 7 is a center point of a left ankle joint in the left ankle joint area, and the eighth keypoint 8 is a center point of a right ankle joint in the right ankle joint area.

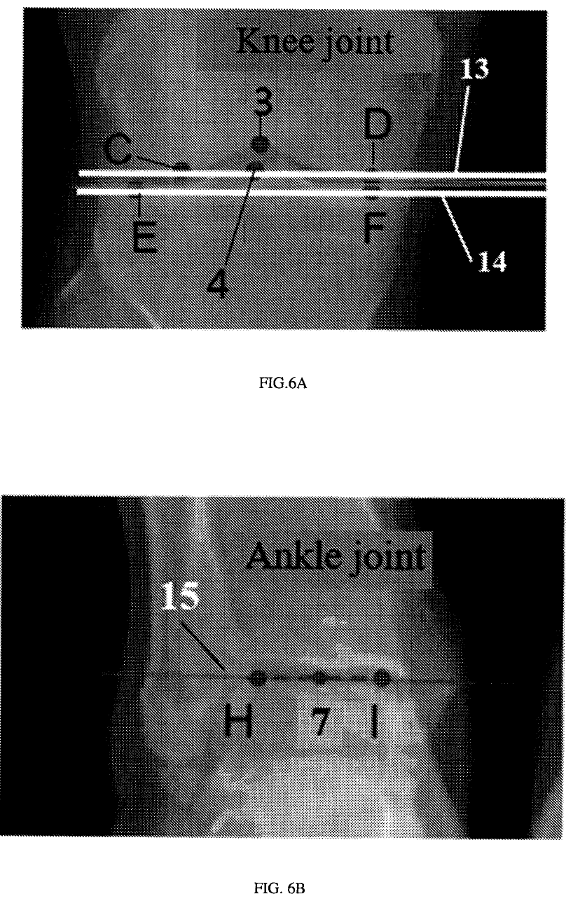

As shown in FIGS. 6A and 6B, 12 joint line keypoints include lowest points C and D of medial and lateral femoral condyles at a distal end of a left femur in the left knee joint area, lowest points C and D (not shown in the figures) of medial and lateral femoral condyles at a distal end of a right femur in the right knee joint area, lowest points E and F of medial and lateral tibial plateaus at a proximal end of a left tibia in the left knee joint area, lowest points E and F (not shown in the figures) of medial and lateral tibial plateaus at a proximal end of a right tibia in the right knee joint area, lowest points H and I of medial and lateral articular surfaces at a distal end of the left tibia in the left ankle joint area, and lowest points H and I (not shown in the figures) of medial and lateral articular surfaces at a distal end of the right tibia in the right ankle joint area respectively.

S202: establishment of characteristic axes

The obtained coordinates of the eight characteristic axis keypoints are connected to draw characteristic axes, and thus four characteristic axes extending along left and right lower limbs are formed, and include a first characteristic axis 9, a second characteristic axis 10, a third characteristic axis 11 and a fourth characteristic axis 12, wherein two endpoints of the first characteristic axis 9 are the first keypoint 1 and the third keypoint 3 respectively, two endpoints of the second characteristic axis 10 are the second keypoint 2 and the fifth keypoint 5 respectively, two endpoints of the third characteristic axis 11 are the fourth keypoint 4 and the seventh keypoint 7 respectively, and two endpoints of the fourth characteristic axis 12 are the sixth keypoint 6 and the eighth keypoint 8 respectively. A left knee joint upper orientation line 13 is formed by connecting the lowest points C and D of the medial and lateral femoral condyles at the distal end of the left femur, a left knee joint lower orientation line 14 is formed by connecting the lowest points E and F of the medial and lateral tibial plateaus at the proximal end of the left tibia, and a left ankle joint orientation line 15 is formed by connecting the lowest points H and I of the medial and lateral articular surfaces at the distal end of the left tibia; and a right knee joint upper orientation line 13, a right knee joint lower orientation line 14 and a right ankle joint orientation line 15 may be obtained by the same method.

Wherein, the first and second characteristic axes correspond to the left and right knee joint upper orientation lines 13, and the third and fourth characteristic axes correspond to the left and right knee joint lower orientation lines 14 and the left and right ankle joint orientation lines 15. As the positions of reference signs in FIGS. 2B, 2C and 2D are the same as those of reference signs in FIG. 2A, the reference signs are only marked on FIG. 2A.

Step 3: angle calculation
slopes among the keypoints are calculated by using the obtained keypoints and characteristic axes, and mechanical lateral distal femoral angles, joint line convergence angles, medial proximal tibial angles and lateral distal tibial angles are calculated by inverse trigonometric functions.

The knee joint orientation lines and the ankle joint lines are keys to this step, and the angle calculation errors can be reduced significantly by decreasing coordinate errors of the points; and considering that the coordinate errors of the keypoints are difficult to decrease, the point coordinates are replaced with the slopes in the present disclosure, and the angles are calculated by an arctan inverse trigonometric function to reduce the angle calculation error obviously.

Where, the angle calculation method is as follows:
the mechanical lateral distal femoral angles (mLDFA) include an included angle between the first characteristic axis 9 and the left distal femoral joint orientation line where the third keypoint 3 is located, and an included angle between the second characteristic axis 10 and the right distal femoral joint orientation line where the fifth keypoint 5 is located; wherein a calculation formula of the included angles is as follows:

$$tan θ = \vert \frac{{k}_{13} - {k}_{CD}}{1 + {k}_{13} {k}_{CD}} \vert = \vert \frac{\frac{{y}_{3} - {y}_{1}}{{x}_{3} - {x}_{1}} - \frac{{y}_{D} - {y}_{C}}{{x}_{D} - {x}_{C}}}{1 + \frac{{y}_{3} - {y}_{1}}{{x}_{3} - {x}_{1}} \cdot \frac{{y}_{D} - {y}_{C}}{{x}_{D} - {x}_{C}}} \vert$$

Where, θ represents the solved angle, k₁₃ represents a slope of the first characteristic axis 9, k_{CD} represents a slope of the left knee joint upper orientation line 13, and x₁ and y₁ represent an abscissa and an ordinate of the first keypoint 1; x₃ and y₃ represent an abscissa and an ordinate of the third keypoint 3; x_{C}, x_{D}, y_{C} and y_{D} represent abscissas and ordinates of two joint line keypoints on the left knee joint upper orientation line 13; and the function is transformed into an arctan that is a reciprocal function according to the above tangent formula, to solve the left mLDFA θ. Correspondingly, the included angle between the second characteristic axis 10 and the right knee joint upper orientation line 13 where the fifth keypoint 5 is located may be solved according to the above formula.

The joint line convergence angles (JLCA) include an included angle between the left knee joint upper orientation line 13 where the third keypoint 3 is located, and the left knee joint lower orientation line 14 where the fourth keypoint 4 is located, and an included angle between the right knee joint upper orientation line 13 where the fifth keypoint 5 is located, and the right knee joint lower orientation line 14 where the sixth keypoint 6 is located; wherein, the calculation of the included angles applies the calculation formula of the mLDFA, that is, the slopes of the two orientation lines are calculated based on the coordinates of the two joint line keypoints on the knee joint upper orientation line 13 and the knee joint lower orientation line 14; after a tan thereof is solved by a trigonometric function, it is transformed into an arctan to solve the left and right joint line convergence angles.

The medial proximal tibial angles (MPTA) include an included angle between the left knee joint lower orientation line 14 where the fourth keypoint 4 is located, and the third characteristic axis 11, and an included angle between the right knee joint lower orientation line 14 where the sixth keypoint 6 is located, and the fourth characteristic axis 12; the calculation of the included angles applies the calculation formula of the above mLDFA, that is, the slope is calculated based on the coordinates of the two endpoints (which are the fourth keypoint 4 and the seventh keypoint 7) of the third characteristic axis 11 and the coordinates of two joint line keypoints on the left knee joint lower orientation line 14; after a tan thereof is solved by the trigonometric function, it is transformed into the arctan to solve the left medial proximal tibial angle; the slope is calculated based on the coordinates of the two endpoints (which are the sixth keypoint 6 and the eighth keypoint 8) of the fourth characteristic axis 12 and the coordinates of two joint line keypoints on the right knee joint lower orientation line 14; and after a tan thereof is solved by the trigonometric function, it is transformed into the arctan to solve the right medial proximal tibial angle.

The lateral distal tibial angles (LDTA) include an included angle between the third characteristic axis 11 and the left ankle joint orientation line 15 where the seventh keypoint 7 is located, and an included angle between the fourth characteristic axis 12 and the right ankle joint orientation line 15 where the eighth keypoint 8 is located. Wherein, the calculation of the included angles applies the calculation formula of the mLDFA, that is, the slope is calculated based on the coordinates of the two endpoints (which are the fourth keypoint 4 and the seventh keypoint 7) of the third characteristic axis 11 and the coordinates of two joint line keypoints on the left knee joint lower orientation line; after a tan thereof is solved by the trigonometric function, it is transformed into the arctan to solve the left medial proximal tibial angle; the slope is calculated based on the coordinates of the two endpoints (which are the sixth keypoint 6 and the eighth keypoint 8) of the fourth characteristic axis 12 and the coordinates of two joint line keypoints on the right knee joint lower orientation line 15; and after a tan thereof is solved by the trigonometric function, it is transformed into the arctan to solve the right medial proximal tibial angle.

Step 4, error analysis and output and storage of results

The function is regarded as a multivariate function θ=arctan (xₙ) according to the above trigonometric formula for calculating the included angle, the function in brackets is set as f, a partial derivative is solved, and the function f is substituted, and simplified to obtain the following error analysis formula of abscissas and ordinates of the keypoints on the third characteristic axis in a single radiograph:

$$\frac{\partial θ}{\partial {x}_{3}} = \frac{1}{1 + {f}^{2}} \frac{\partial f}{\partial {x}_{3}} \\ \frac{\partial θ}{\partial {y}_{3}} = \frac{1}{1 + {f}^{2}} \frac{\partial f}{\partial {y}_{3}}$$

Where, x₃ is an abscissa of a characteristic axis keypoint, and y₃ is an ordinate of a characteristic axis keypoint. Therefore, the error values of the third characteristic axis keypoints are obtained; the error values of the other characteristic axis keypoints are obtained according to the above formula in sequence; the above steps are repeated till error values of all input radiographs are obtained, and the plurality of error values serve as statistical analysis results of errors to be drawn into a line chart as shown in FIG. 3; the mLDFA, JLCA, MPTA, LDTA and a plurality of characteristic axes are shown on the radiographs, and the measurement results are shown in a picture output form; and the angles are recorded in Excel. According to the output results serving as a judgment basis, diagnostic results of the deformity are output through classification and reasoning of an expert system, so as to provide a reasonable and precise correction method for patients.

Although the functions and working process of the present disclosure are described with reference to accompanying drawings above, the present disclosure is not limited to the above specific functions and working process. The above specific implementations are only exemplary instead of restrictive. Those ordinarily skilled in the art may also make many forms without departing from the purpose of the present disclosure and the scope protected by claims under inspiration of the present disclosure, and these forms all fall within the protection of the present disclosure.

1. A measurement and diagnostic system for weight-bearing lines of both lower limbs, comprising:
a data loading module, configured to load and read a data file (.mat) of keypoint position information of training sets and radiographs to be measured, and select joint image positions in each radiograph;
a keypoint selection module, configured to classify and identify the joint image positions selected by the data loading module through a convolutional neural network by taking obtained joint images as input layers, to obtain coordinates of corresponding keypoints on the radiographs, comprising coordinates of eight characteristic axis keypoints and coordinates of a plurality of joint line keypoints; connect the obtained coordinates of the eight keypoints to draw characteristic axes, thereby forming four characteristic axes extending along left and right lower limbs, comprising a first characteristic axis (9), a second characteristic axis (10), a third characteristic axis (11) and a fourth characteristic axis (12); and connect the coordinates of the joint line keypoints to draw six joint lines, thereby forming left and right knee joint upper orientation lines (13), left and right knee joint lower orientation lines (14), and left and right ankle joint orientation lines (15), wherein the first and second characteristic axes correspond to the left and right knee joint upper orientation lines (13) respectively, and the third and fourth characteristic axes correspond to the left and right knee joint lower orientation lines (14) and the left and right ankle joint orientation lines (15) respectively;
an angle calculation module, configured to calculate slopes among the keypoints by using the obtained keypoints, characteristic axes and joint lines, and calculate mechanical lateral distal femoral angles, joint line convergence angles, medial proximal tibial angles and lateral distal tibial angles by inverse trigonometric functions; and
a mechanical axis diagnosis and output module, configured to compare the obtained angle values with preset reference thresholds of the mechanical lateral distal femoral angles, the joint line convergence angles, the medial proximal tibial angles and the lateral distal tibial angles according to the angles obtained by the angle calculation module, wherein if the obtained angle values are not within the reference thresholds, it indicates that the angles are abnormal, and there is possibly a lower limb deformity, and if all the angle values are within the reference thresholds, it indicates that there is no lower limb deformity; and then, output the obtained angle values, show the plurality of characteristic axes and joint lines on the radiographs, and represent measurement results in a picture output form.

2. The measurement and diagnostic system for the weight-bearing lines of both lower limbs according to claim 1, wherein the preset reference thresholds of the mechanical lateral distal femoral angles, the joint line convergence angles, the medial proximal tibial angles and the lateral distal tibial angles are as follows: the reference threshold is 85° to 90° for the mechanical lateral distal femoral angles, 0° to 2° for the joint line convergence angles, 85° to 90° for the medial proximal tibial angles, and 86° to 92° for the lateral distal tibial angles.

3. The measurement and diagnostic system for the weight-bearing lines of both lower limbs according to claim 1, wherein the joint images include a left hip joint area, a right hip joint area, a left knee joint area, a right knee joint area, a left ankle joint area and a right ankle joint area;
the characteristic axis keypoints comprise a first keypoint (1), a second keypoint (2), a third keypoint (3), a fourth keypoint (4), a fifth keypoint (5), a sixth keypoint (6), a seventh keypoint (7) and an eighth keypoint (8);
the first keypoint (1) is a center point of a left femoral head in the left hip joint area, the second keypoint (2) is a center point of a right femoral head in the right hip joint area, the third keypoint (3) is a center point of a left femoral knee joint in the left knee joint area, the fourth keypoint (4) is a center point of a left tibial knee joint in the left knee joint area, the fifth keypoint (5) is a center point of a right femoral knee joint in the right knee joint area, the sixth keypoint (6) is a center point of a right tibial knee joint in the right knee joint area, the seventh keypoint (7) is a center point of a left ankle joint in the left ankle joint area, and the eighth keypoint (8) is a center point of a right ankle joint in the right ankle joint area;
wherein two endpoints of the first characteristic axis (9) are the first keypoint (1) and the third keypoint (3) respectively, two endpoints of the second characteristic axis (10) are the second keypoint (2) and the fifth keypoint (5) respectively, two endpoints of the third characteristic axis (11) are the fourth keypoint (4) and the seventh keypoint (7) respectively, and two endpoints of the fourth characteristic axis (12) are the sixth keypoint (6) and the eighth keypoint (8) respectively.

4. The measurement and diagnostic system for the weight-bearing lines of both lower limbs according to claim 1, wherein the joint line keypoints comprise lowest points of medial and lateral femoral condyles at a distal end of a left femur in the left knee joint area, lowest points of medial and lateral femoral condyles at a distal end of a right femur in the right knee joint area, lowest points of medial and lateral tibial plateaus at a proximal end of a left tibia in the left knee joint area, lowest points of medial and lateral tibial plateaus at a proximal end of a right tibia in the right knee joint area, lowest points of medial and lateral articular surfaces at a distal end of the left tibia in the left ankle joint area, and lowest points of medial and lateral articular surfaces at a distal end of the right tibia in the right ankle joint area;
the left and right knee joint upper orientation lines (13), the left and right knee joint lower orientation lines (14), and the left and right ankle joint orientation lines (15) are obtained as follows:
the left knee joint upper orientation line (13) is a connecting line between two joint line keypoints located at the distal end of the left femur;
the right knee joint upper orientation line (13) is a connecting line between two joint line keypoints located at the distal end of the right femur;
the left knee joint lower orientation line (14) is a connecting line between two joint line keypoints located at the proximal end of the left tibia;
the right knee joint lower orientation line (14) is a connecting line between two joint line keypoints located at the proximal end of the right tibia;
the left ankle joint orientation line (15) is a connecting line between two joint line keypoints located at the distal end of the left tibia; and
the right ankle joint orientation line (15) is a connecting line between two joint line keypoints located at the distal end of the right tibia.

5. The measurement and diagnostic system for the weight-bearing lines of both lower limbs according to claim 1, wherein the keypoint position training module is configured to train a keypoint model by a target detection algorithm model in advance, to obtain specific positions of all keypoints in the radiographs; and the keypoint position information is saved by the training model properly trained in a .mat format for extracting keypoints subsequently.

6. An intelligent measurement method for weight-bearing lines of both lower limbs, comprising the following steps:
step 1, importing a plurality of human lower limb full-length digital radiographs to be measured, loading and reading keypoint position training models pre-trained properly, and selecting joint image positions in each radiograph, wherein the joint images comprise a left hip joint area, a right hip joint area, a left knee joint area, a right knee joint area, a left ankle joint area and a right ankle joint area;
step 2, extracting keypoints by a convolutional neural network method
performing classification and identification through a convolutional neural network by taking the joint images obtained in step 1 as input layers, to obtain coordinates of corresponding keypoints on the radiographs, comprising coordinates of eight characteristic axis keypoints and coordinates of a plurality of joint line keypoints; connecting the obtained coordinates of the eight keypoints to draw characteristic axes, thereby forming four characteristic axes extending along left and right lower extremities, comprising a first characteristic axis (9), a second characteristic axis (10), a third characteristic axis (11) and a fourth characteristic axis (12); and connecting the coordinates of the joint line keypoints to draw six joint lines, thereby forming left and right knee joint upper orientation lines (13), left and right knee joint lower orientation lines (14), and left and right ankle joint orientation lines (15), wherein the first and second characteristic axes correspond to the left and right knee joint upper orientation lines respectively, and the third and fourth characteristic axes correspond to the left and right knee joint lower orientation lines, and the left and right ankle joint orientation lines respectively;
the characteristic axis keypoints comprise a first keypoint (1), a second keypoint (2), a third keypoint (3), a fourth keypoint (4), a fifth keypoint (5), a sixth keypoint (6), a seventh keypoint (7) and an eighth keypoint (8);
step 3, calculating slopes among the keypoints by using the obtained keypoints, characteristic axes and joint lines, and calculating mechanical lateral distal femoral angles, joint line convergence angles, medial proximal tibial angles and lateral distal tibial angles by inverse trigonometric functions; wherein
the mechanical lateral distal femoral angles comprise an included angle between the first characteristic axis (9) and the left knee joint upper orientation line (13) where the third keypoint (3) is located, and an included angle between the second characteristic axis (10) and the right knee joint upper orientation line (13) where the fifth keypoint (5) is located; a calculation formula of the included angles is as follows:
$$tan θ = \vert \frac{{k}_{13} - {k}_{CD}}{1 + {k}_{13} {k}_{CD}} \vert = \vert \frac{\frac{{y}_{3} - {y}_{1}}{{x}_{3} - {x}_{1}} - \frac{{y}_{D} - {y}_{C}}{{x}_{D} - {x}_{C}}}{1 + \frac{{y}_{3} - {y}_{1}}{{x}_{3} - {x}_{1}} \cdot \frac{{y}_{D} - {y}_{C}}{{x}_{D} - {x}_{C}}} \vert$$
where, θ represents the solved angle, k₁₃ represents a slope of the first characteristic axis (9), k_{CD} represents a slope of the left knee joint upper orientation line (13), and x₁ and y₁ represent an abscissa and an ordinate of the first keypoint (1); x₃ and y₃ represent an abscissa and an ordinate of the third keypoint (3); x_{C}, x_{D}, y_{C} and y_{D} represent abscissas and ordinates of two joint line keypoints of the left knee joint upper orientation line (13); the function is transformed into an arctan that is a reciprocal function according to the above tangent formula, to solve the left mechanical lateral distal femoral angle; correspondingly, the included angle between the second characteristic axis (10) and the right knee joint upper orientation line (13) where the fifth keypoint (5) is located may be obtained according to the above formula;
the joint line convergence angles comprise an included angle between the left knee joint upper orientation line (13) where the third keypoint (3) is located, and the left knee joint lower orientation line (14) where the fourth keypoint (4) is located, and an included angle between the right knee joint upper orientation line (13) where the fifth keypoint (5) is located, and the right knee joint lower orientation line (14) where the sixth keypoint (6) is located; the calculation of the included angles applies the calculation formula of the mechanical lateral distal femoral angles;
the medial proximal tibial angles comprise an included angle between the left knee joint lower orientation line (14) where the fourth keypoint (4) is located, and the third characteristic axis (11), and an included angle between the right knee joint lower orientation line (14) where the sixth keypoint (6) is located, and the fourth characteristic axis (12); the calculation of the included angles applies the calculation formula of the mechanical lateral distal femoral angles;
the lateral distal tibial angles comprise an included angle between the third characteristic axis (11) and the left ankle joint orientation line (15) where the seventh keypoint (7) is located, and an included angle between the fourth characteristic axis (12) and the right ankle joint orientation line (15) where the eighth keypoint (8) is located; the calculation of the included angles applies the calculation formula of the mechanical lateral distal femoral angles;
step 4, outputting and storing results showing the angle values, the plurality of characteristic axes and the joint lines obtained step 3 on the radiographs, and representing measurement results in a picture output form.

7. The intelligent measurement method for the weight-bearing lines of both lower limbs according to claim 6, wherein the keypoint position training module is configured to train a keypoint model by a target detection algorithm model in advance, to obtain specific positions of all keypoints in pictures; and the keypoint position information is saved by the training model properly trained in a .mat format for extracting keypoints subsequently.

8. The intelligent measurement method for the weight-bearing lines of both lower limbs according to claim 6, wherein the step 4 further comprises analyzing errors of the angle values obtained in step 3, specifically comprising:
regarding it as a multivariate function θ=arctan (xₙ) according to a trigonometric formula for calculating the included angles, letting the function in brackets be f, solving a partial derivative, substituting the function f, and simplifying it to obtain the following error analysis formula of abscissas and ordinates of characteristic axis keypoints on a single radiograph:
$$\frac{\partial θ}{\partial {x}_{n}} = \frac{1}{1 + {f}^{2}} \frac{\partial f}{\partial {x}_{n}} \\ \frac{\partial θ}{\partial {y}_{n}} = \frac{1}{1 + {f}^{2}} \frac{\partial f}{\partial {y}_{n}}$$
where, xₙ is an abscissa of a characteristic axis keypoint, and yₙ is an ordinate of the characteristic axis keypoint; and
error values of the characteristic axis keypoints are obtained according to the above formula in sequence; and statistical analysis results of the error values obtained from a plurality of radiographs are drawn into a line chart for analysis by users.

9. The intelligent measurement method for the weight-bearing lines of both lower limbs according to claim 6, wherein the step 4 further comprises recording the angle values obtained in step 3 in pre-initialized Excel.
